(19) United States
(12) Patent Application Publication
(10) Pub. No.: US 2026/0295288 A1
(43) Pub. Date: Oct. 1, 2026

(54) HYBRID METAL HALIDE SEMICONDUCTORS AS NOVEL PHOTOTHERAPEUTIC TREATMENTS AGAINST PULMONARY CANCER

(71) Applicant: UNIVERSITY OF SOUTH FLORIDA, Tampa, FL (US)

(72) Inventors: Ioannis SPANOPOULOS, Tampa, FL (US); Alissa ANDERSON, Tampa, FL (US)

(21) Appl. No.: 19/578,760

(22) Filed: Mar. 25, 2026

Related U.S. Application Data
(60) Provisional application No. 63/777,380, filed on Mar. 25, 2025.

(51) Int. Cl. A61N 5/06

(52) U.S. Cl. CPC A61N 5/0613; A61N 2005/0632; A61N 2005/065

(57) ABSTRACT

Disclosed herein are water-stable, lead-free metal halide semiconductor materials comprising bismuth halide frameworks and phosphonium-based organic components, as well as methods of making and using the same for therapeutic applications. The materials exhibit intrinsic photo-responsive and redox-active properties under visible light irradiation, enabling controlled generation of reactive oxygen species and selective cytotoxic activity toward cancerous cells, including pulmonary tumors, while limiting off-target effects. The incorporation of phosphonium ligands enhances charge-transfer behavior and stabilizes the metal center under physiological conditions, resulting in improved photo-activated redox performance. These hybrid materials provide a tunable platform for light-controlled or redox-mediated therapies and represent a new class of biocompatible, lead-free semiconductor agents for anticancer treatment and related biomedical applications.

### CROSS REFERENCE TO RELATED APPLICATIONS

This application claims the benefit of and priority to U.S. provisional application Ser. No. 63/777,380 entitled “HYBRID METAL HALIDE SEMICONDUCTORS AS NOVEL PHOTOTHERAPEUTIC TREATMENTS AGAINST PULMONARY CANCER”, filed Mar. 25, 2025, the entire disclosure of which is hereby incorporated by reference.

### FIELD

The present disclosure relates to hybrid organic-inorganic semiconductor materials, including lead-free bismuth halide semiconductors with organic phosphonium ligands and phosphonium components, and to therapeutic compositions and phototherapeutic treatments against pulmonary cancer that use such materials as reactive oxygen species (ROS) generators in the visible-NIR region.

### BACKGROUND

Pulmonary cancer is a major cause of cancer-related deaths, and current treatment options is limited by systemic toxicity, invasiveness, and significant side effects. Phototherapeutic treatments provide controllable, localized, and selective treatment methods in response to light, and is less invasive with fewer side effects. There remains a need for potent antitumor agents composed of biocompatible elements that are water-stable and capable of generating reactive oxygen species in biologically relevant environments.

Hybrid metal halide semiconductors with tailorable optoelectronic properties support localized inhibition of tumor cells by enabling controlled reactive oxygen species generation under visible-light irradiation.

### SUMMARY

Disclosed herein are lead-free bismuth halide semiconductor materials including zero-dimensional hybrid structures formed from face-sharing Bi₂I₉ dimers separated by organic phosphonium ligands. The incorporation of phosphonium components provides structural stability and enhances charge-transfer behavior. The materials exhibit optical absorption and emission in the visible-NIR region and are light-active and redox-active, generating reactive oxygen species through redox-mediated processes upon irradiation.

The reactive oxygen species include Type I reactive oxygen species, including superoxide, hydroxyl radicals, hydroperoxyl radicals, and hydrogen peroxide. Disclosed herein are therapeutic compositions comprising the materials in an aqueous medium, including water. In some embodiments, the disclosure provides phototherapeutic treatments against pulmonary cancer, including pulmonary cancer strain A549 and non-small cell lung cancer, including photoactivation using clinically relevant light sources to provide localized cytotoxic effects within nanometer-scale regions and selective inhibition of cancer cells while minimizing damage to surrounding healthy tissue.

### BRIEF DESCRIPTION OF THE DRAWINGS

FIG. 1A shows representative crystal structures of a zero-dimensional phosphonium-bismuth iodide material viewed along a (showing the b-c plane). FIG. 1B shows representative crystal structures of a zero-dimensional phosphonium-bismuth iodide material viewed along c (showing the a-b plane). FIG. 1C shows representative crystal structures of a zero-dimensional phosphonium-bismuth iodide material viewed along b (showing the a-c plane), as indicated by the axis triads. FIG. 1D shows a PDod ligand.

FIG. 2 shows representative powder X-ray diffraction patterns demonstrating phase purity and water stability after aqueous exposure.

FIG. 3A shows representative thermogravimetric analysis of (PDod)₃Bi₂I₉.

FIG. 3B shows differential scanning calorimetry results of (PDod)₃Bi₂I₉.

FIG. 4 shows representative diffuse reflectance spectra of (PDod)₃Bi₂I₉.

FIG. 5A shows representative cyclic voltammetry results under irradiation and in the dark. FIG. 5B shows a Nyquist plot under irradiation and in the dark.

FIG. 6A shows representative degradation assays of (PDod)₃Bi₂I₉, showing Type I ROS for TMPD (TMPD=N,N,N′,N′-tetramethyl-p-phenylenediamine). FIG. 6B shows representative degradation assays of (PDod)₃Bi₂I₉, showing Type I ROS for ABDA (ABDA=9,10-anthracenediyl-bis(methylene)dimalonic acid). FIG. 6C shows representative degradation assays of (PDod)₃Bi₂I₉, showing Type I ROS for RhB (RhB=Rhodamine B). FIG. 6D shows representative degradation assays of (PDod)₃Bi₂I₉, showing Type I ROS for MB (MB=Methylene blue).

FIG. 7 shows a representative dose-response curve evidencing cytotoxicity against a pulmonary cancer strain A549.

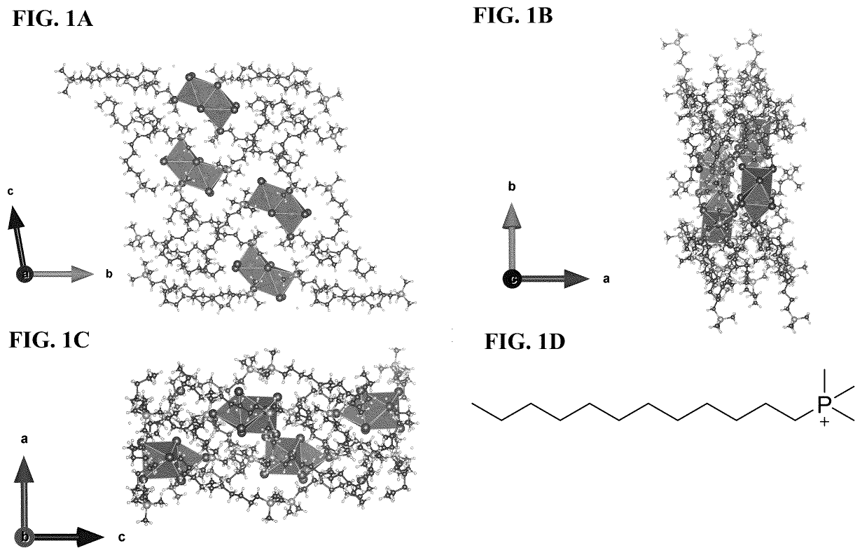

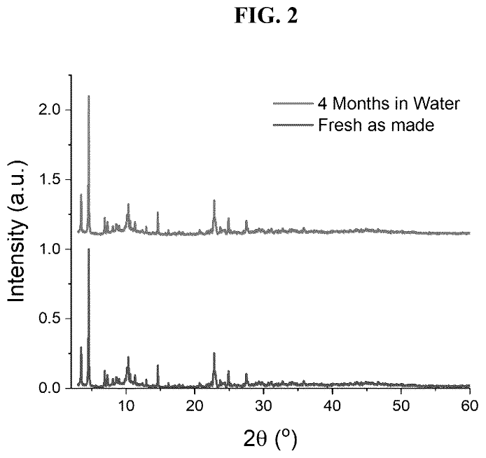

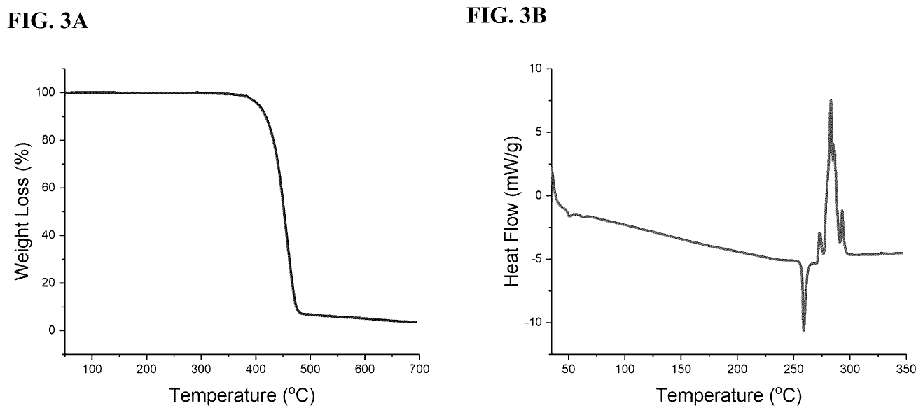

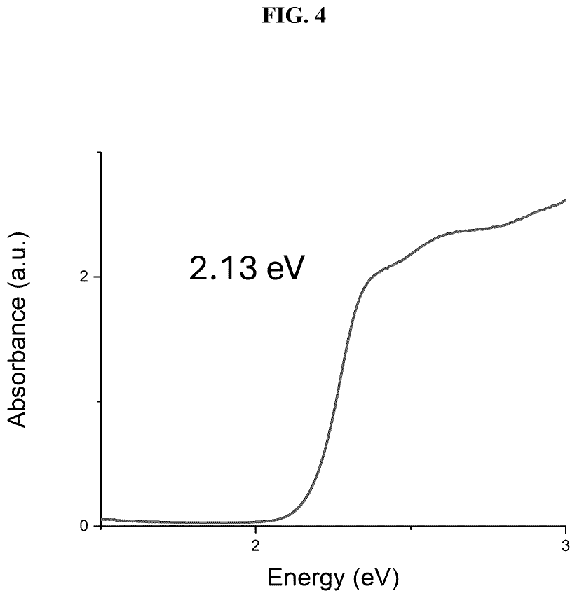

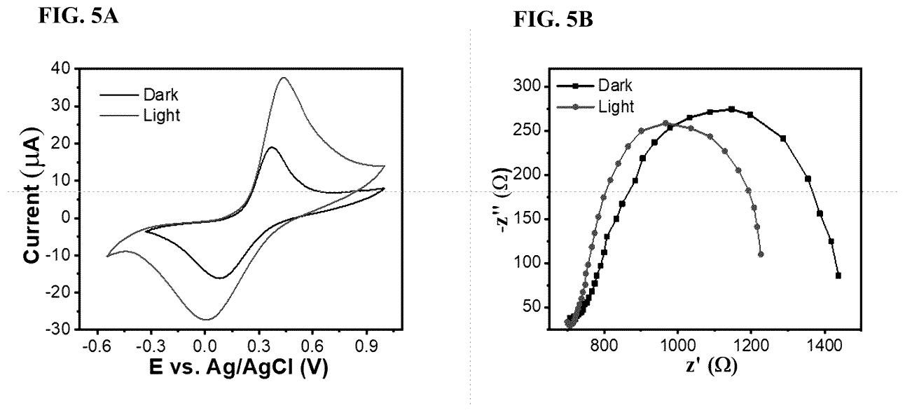

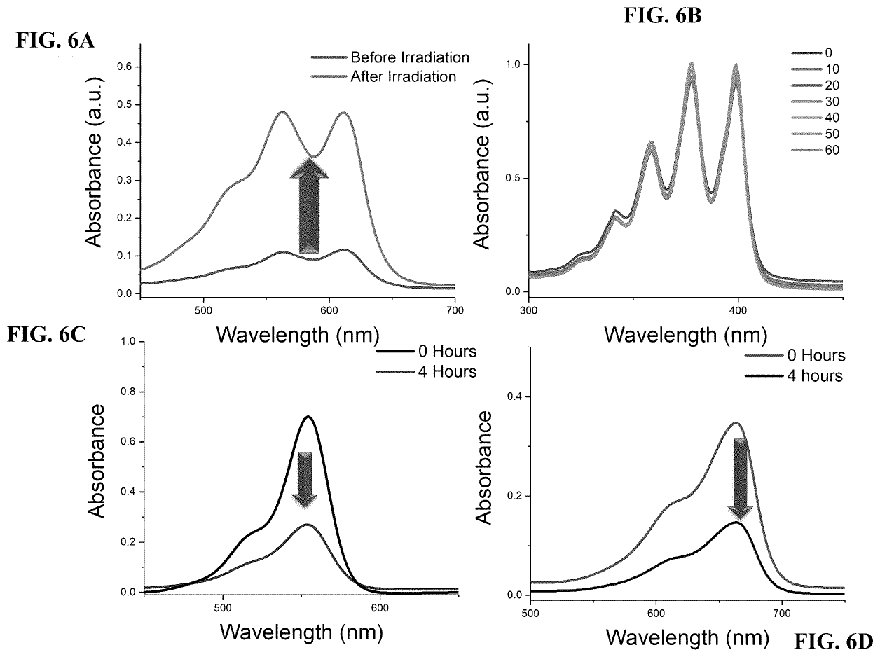

### DEFINITIONS

As used herein, the term “Visible-NIR” refers to wavelengths in the visible to near-infrared region, from about 400 nm to about 900 nm.

As used herein, the term “near-infrared” refers to wavelengths from about 700 nm to about 2500 nm.

As used herein, the term “Reactive oxygen species” (ROS) refers to oxygen radicals and related species, and includes Type I reactive oxygen species formed through electron-transfer reactions and Type II reactive oxygen species formed through energy-transfer reactions, including singlet oxygen.

As used herein, the term “Type I reactive oxygen species” refers to reactive oxygen species formed through electron transfer reactions and includes superoxide, hydroxyl radicals, hydroperoxyl radicals, and hydrogen peroxide.

As used herein, the term “Type II reactive oxygen species” refers to reactive oxygen species formed through energy-transfer reactions and includes singlet oxygen.

As used herein, the term “Water-stable” refers to materials that retain phase identity and crystallinity after exposure to water under specified conditions, as determined by powder X-ray diffraction.

As used herein, the term “Pulmonary cancer” refers to pulmonary cancer strains and non-small cell lung cancer.

As used herein, “X” is a halide selected from iodide, bromide, chloride, and combinations thereof.

As used herein, “PDod” refers to an organic phosphonium ligand/cationic component used in the disclosed lead-free bismuth halide semiconductor materials, including the phosphonium component present in (PDod)₃Bi₂I₉, and includes substituted variants thereof.

As used herein, the term “illumination” refers to exposure of a target to non-ionizing electromagnetic radiation that includes visible light and near-infrared light.

As used herein, the term “irradiation” refers to exposure to γ-rays and x-rays and exposure of a target to electromagnetic radiation, including visible light and near-infrared light, delivered by a light source such as an LED, laser, or fiber-optic emitter.

As used herein, “optoelectronic properties” refers to the optical and electronic characteristics of a material, including light absorption/emission behavior and charge-carrier generation and transport behavior.

### DETAILED DESCRIPTION OF THE EMBODIMENTS

Hybrid organic-inorganic bismuth halide semiconductors described herein combine an inorganic bismuth-halide framework with organic phosphonium cations. In some embodiments, the inorganic framework comprises face-sharing BiX₆ octahedra arranged as Bi₂X₉³⁻ dimers.

In some embodiments, the materials are zero-dimensional, such that the dimers are spatially separated and charge-balanced by phosphonium cations. Such structural features contribute to stability in aqueous environments and to photoinduced charge-carrier generation.

In some embodiments, a material comprises an inorganic bismuth halide framework including Bi₂X₉³⁻ dimers and an organic phosphonium cationic component that charge-balances the framework.

In some embodiments, X is a halide, and each X in BiX₆ is independently selected from iodide, bromide, chloride, and combinations thereof.

In some embodiments, the phosphonium cation comprises at least one C₄-C₂₀ alkyl substituent.

In some embodiments, the material is crystalline and exhibits an optical absorption edge corresponding to a bandgap in a range from about 1.7 eV to about 3 eV.

In some embodiments, the material is crystalline and exhibits an optical absorption edge corresponding to a bandgap in a range from 1.3 to 2.0 eV, from 1.5 to 2.3 eV, from 1.8 to 2.6 eV and from 1.7 to 2.6 eV.

In some embodiments, the material remains crystalline after immersion in liquid water for at least 4 months.

In some embodiments, the material remains crystalline after immersion in liquid water for at least 1-2 months, 2-3 months, 3 to 4 months and 1 to 5 months.

In some embodiments, the material is represented by (R₃R′P)₃Bi₂X₉, wherein each R₃R′P⁺ is independently an alkylarylphosphonium cation, a trialkylphosphonium cation, a triarylphosphonium cation, or a substituted variant thereof, and X is a halide.

In some embodiments, the material comprises (PDod)Bi₂X₉, where PDod is a phosphonium ligand bearing dodecyl substituents.

In some embodiments, the materials exhibit strong optical absorption and emission in the visible to near-infrared region, enabling activation with clinically relevant light sources. These absorption and emission characteristics define optoelectronic properties of the materials and support photoactivation under visible to near-infrared light to increase reactive oxygen species generation.

In some embodiments, cyclic voltammetry under irradiation exhibits increased anodic and cathodic current compared to dark conditions, consistent with photoexcited charge-carrier generation.

In some embodiments, electrochemical impedance spectroscopy under irradiation shows reduced charge-transfer resistance relative to dark conditions, supporting enhanced interfacial electron dynamics.

In some embodiments, the materials are redox-active and generate reactive oxygen species (ROS) in aqueous media.

In some embodiments, the ROS comprises Type I species including superoxide, hydroxyl radical, hydroperoxyl radical, and hydrogen peroxide.

In some embodiments, Type I ROS generation is evidenced by oxidation of redox-responsive dyes (for example, TMPD) under visible-light exposure, with minimal change in singlet-oxygen probes (for example, ABDA), consistent with a predominantly Type I pathway.

In some embodiments, a pharmaceutical composition comprises any of the materials described herein and a pharmaceutically acceptable carrier.

In some embodiments, the material is formulated as nanoparticles, microparticles, crystalline particles, a suspension, or a dispersion.

In some embodiments, the carrier comprises water, buffered aqueous solutions, saline, dextrose solutions, or biocompatible excipients suitable for inhalation, intratumoral injection, intravenous injection, intrapleural administration, or combinations thereof.

In some embodiments, the composition further comprises one or more stabilizers, surfactants, or tonicity agents.

In some embodiments, methods are provided for treating pulmonary cancer in a subject.

In some embodiments, the method comprises administering to the subject an effective amount of a pharmaceutical composition comprising a disclosed material.

In some embodiments, the pulmonary cancer comprises non-small cell lung cancer.

In some embodiments, administration results in selective cytotoxicity toward pulmonary cancer cells via redox-mediated generation of reactive oxygen species.

In some embodiments, the method is performed without irradiating the subject with therapeutic light.

In some embodiments, the method further comprises irradiating a target tissue with visible or near-infrared light to increase ROS generation at the target tissue.

In some embodiments, irradiation comprises delivering light having a wavelength from about 450 nm to about 900 nm, in one or more fractions.

In some embodiments, the light source comprises LEDs, lasers, fiber-optic delivery systems, endoscopic light delivery systems, or combinations thereof.

In some embodiments, a method of making a disclosed material comprises reacting a bismuth(III) source with a hydrohalic acid HX (where X is iodide, bromide, or chloride) in a solvent and in the presence of an organic phosphonium salt to form a precipitate comprising the material, and isolating the precipitate.

In some embodiments, the bismuth(III) source comprises Bi₂O₃, BiX₃, Bi(NO₃)₃, Bi(OAc)₃, or combinations thereof.

In some embodiments, the solvent comprises water, alcohols, polar aprotic solvents, or mixtures thereof.

In some embodiments, a kit comprises: (i) a container comprising a pharmaceutical composition that includes a disclosed material; and (ii) instructions for administering the pharmaceutical composition to a subject having pulmonary cancer, with optional irradiation of a target tissue with visible or near-infrared light.

In some embodiments, the kit further comprises one or more additional containers containing diluents, buffers, syringes, nebulizers, or light-delivery components.

In some embodiments, the as-prepared crystals are reddish-orange single crystals.

In some embodiments, single-crystal X-ray diffraction reveals a triclinic structure (space group P-1) comprising face-sharing Bi₂I₉ dimers separated and charge-balanced by phosphonium cations.

In some embodiments, Bi—I bond lengths fall into two groups: (i) from about 2.89608 Å to about 2.96019 Å for non-bridging iodides and (ii) from about 3.21246 Å to about 3.337154 Å for bridging iodides.

In some embodiments, I—Bi—I bond angles range from about 79.3280 to about 172.84750.

In some embodiments, the stereochemically active 6s² lone pair of electrons in bismuth enables formation of bismuth-halide frameworks with a broad range of nuclearities and coordination environments, thereby supporting diverse semiconductor architectures within the disclosed material class.

In some embodiments, bismuth-based semiconductors exhibit band-edge positions that support efficient separation of photogenerated electron-hole pairs under visible-light exposure, and a bismuth-derived conduction band (including 6p orbital character) participates in electron-transfer reactions that contribute to Type I reactive oxygen species generation.

In some embodiments, the organic phosphonium cationic component acts as a σ-donor and π-acceptor ligand environment that stabilizes the bismuth center and enhances charge-transfer transitions, thereby improving photocatalytic performance and/or ROS generation under visible-light exposure.

In some embodiments, inhibition of biological cells by a disclosed semiconductor material proceeds by one or more mechanisms selected from: (i) induction of oxidative stress by reactive oxygen species that interact with cellular structures, (ii) release of metal species that interfere with genetic material, and (iii) a nonoxidative mechanism in which cellular metabolism is reduced without inducing oxidative stress.

In some embodiments, exposure to reactive oxygen species causes damage to cellular phospholipids, proteins, and nucleic acids, thereby contributing to loss of viability and/or lysis of target cells.

In some embodiments, the disclosed material acts as a catalytic redox agent that promotes redox cycling and/or catalytic generation of reactive oxygen species under physiological conditions, rather than functioning solely as a photosensitizer.

### EXAMPLES

### Example 1: Synthesis of a Phosphonium-Bismuth Iodide Semiconductor

High-quality crystals of (PDod)3Bi₂I₉ were prepared by reacting a bismuth (III) oxide source with a Trid linker in a hydroiodic acid (HI) solution in the presence of a phosphonium ligand/salt, followed by precipitation and isolation of the resulting solid. Crystals were obtained upon controlled cooling after heating in hydroiodic acid solution. Structural analysis by single-crystal X-ray diffraction indicated a zero-dimensional structure comprising face-sharing Bi₂I₉ dimers separated by phosphonium cations.

### Example 2: Water Stability and Phase Purity

Fresh crystals were immersed in liquid water for an extended period and subsequently analyzed by powder X-ray diffraction. the post-immersion diffraction pattern remains substantially consistent with the fresh material, indicating retention of phase identity and crystallinity after aqueous exposure for at least 4 months. In-house powder X-ray diffraction (PXRD) confirmed phase purity, where an experimental PXRD pattern was substantially identical to a calculated pattern derived from single-crystal X-ray diffraction.

### Example 2A: Morphology and Elemental Analysis (SEM/EDS)

Scanning electron microscopy (SEM) was performed to evaluate crystal morphology. SEM revealed a needle-like crystal shape. Energy-dispersive X-ray spectroscopy (EDS) was performed to confirm elemental composition. EDS confirmed a bismuth-to-iodine ratio of about 1:4.39, consistent with the expected stoichiometry for (PDod)3Bi₂I₉.

### Example 3: Optical and Thermal Characterization

UV-vis diffuse reflectance spectroscopy was performed and analyzed using the Kubelka-Munk function to determine an optical absorption edge. (PDod)3Bi₂I₉ exhibits a sharp absorption edge corresponding to a bandgap of about 2.13 eV. Thermogravimetric analysis further showed one degradation step, where a sharp weight loss was attributed to loss of inorganic groups. Thermogravimetric analysis indicated thermal stability up to about 400° C. Differential scanning calorimetry (DSC) demonstrated two apparent phase changes in a temperature range from about 25° C. to about 350° C., including an endothermic feature at about 200° C. followed by one or more exothermic features.

### Example 4: Photoelectrochemical Response

Cyclic voltammetry was conducted under both light and dark conditions. Cyclic voltammetry profiles showed a pronounced increase in anodic and cathodic current under white light irradiation, and a notable decrease in current response relative to focused white light, consistent with photoinduced charge carrier excitation and enhanced electron transfer. Current response (CR) increased under light, consistent with photoexcited charge carriers. Electrochemical impedance spectroscopy showed reduced charge-transfer resistance under illumination, consistent with enhanced interfacial electron transfer. Electrochemical impedance spectroscopy shows a decrease in charge transfer resistance (Rct) under illumination relative to dark conditions, indicating faster interfacial electron transport upon light exposure and optoelectronic properties associated with photoexcited carrier generation and interfacial charge transfer.

### Example 5: Type I Reactive Oxygen Species Generation

ROS generation was evaluated in abiotic dye assays using aqueous suspensions of the material. Oxidation of TMPD occurred within about 90 seconds of visible-light exposure, as evidenced by emergence of absorbance peaks around 563 nm and 611 nm. Singlet-oxygen probe assays using ABDA showed minimal change under comparable conditions, consistent with a predominantly Type I pathway. Additional assays using methylene blue and rhodamine B showed dye degradation consistent with radical-mediated oxidation. Methylene blue (MB) degradation was monitored at 664 nm. Rhodamine B (RhB) exhibited a characteristic absorption band centered at about 554 nm, and the intensity decreased following irradiation, consistent with progressive dye degradation. Dye assays were performed in triplicate in pure water under ambient visible light without addition of oxidants (for example, hydrogen peroxide) or sacrificial donors to simulate biologically relevant conditions. ABDA showed minimal change after about 60 minutes of irradiation under comparable conditions.

### Example 5A: Photodegradation Kinetics

MB degradation followed pseudo-first-order kinetics with k=0.0034 min⁻¹ and R2=0.9936, and RhB degradation followed pseudo-first-order kinetics with k=0.0033 min⁻¹ and R2=0.984. Corresponding half-lives were about 204 minutes for MB and about 210 minutes for RhB. Pseudo-first-order kinetics were evaluated using Equation (2):

$$ln ( \frac{{C}_{0}}{{C}_{t}} ) = {K}_{a} t$$

where Ct is the dye concentration at time t, C₀ is the initial dye concentration, and K_{α} is a rate constant (min⁻¹). Photodegradation kinetics for methylene blue (MB) and rhodamine B (RhB) were evaluated using pseudo-first-order kinetics. Percentage degradation was calculated using Equation (1):

$$\% degredation = \frac{{C}_{0} - C}{{C}_{0}}$$

where C₀ is the initial dye concentration and C is the final dye concentration.

### Example 6: Cytotoxicity Toward Pulmonary Cancer Cells

Cytotoxicity was evaluated in vitro against a pulmonary cancer cell line (for example, A549) using a dose-response assay. (PDod)3Bi₂I₉ exhibited an IC₅₀ in the microgram per milliliter range, evidencing potent inhibitory activity. selective activity is observed against cancer cells with minimal activity against non-target microbial species under dark conditions.

TABLE 1
IC₅₀ of (PDod)3Bi₂I₉ against various species.

| | |
|---|---|
| Species | IC₅₀ |

| | | |
|---|---|---|
| Pulmonary Cancer A549 | 2.118 ± 1.4 | μg/ml |
| Escherichia Coli ATCC 25922 | >10 | μM |
| Pseudomonas aeruginosa ATCC 27853 | >10 | μM |
| Klebsiella pneumoniae ATCC 13383 | >10 | μM |
| Staphylococcus aureus ATCC 33591 | >10 | μM |
| Staphylococcus epidermidis RP62A | >10 | μM |
| Enterococcus faecalis ATCC 700802 | >10 | μM |
| Candida albicans MYA-2876 | >10 | μM |
| Candida albicans ATCC 18804 | >10 | μM |
| Candida albicans ATCC 28121 | >10 | μM |
| Candida albicans ATCC 76458 | >10 | μM |
| Candida albicans ATCC 90029 | >10 | μM |
| Candidozyma auris AR-0385 | >10 | μM |

### Results

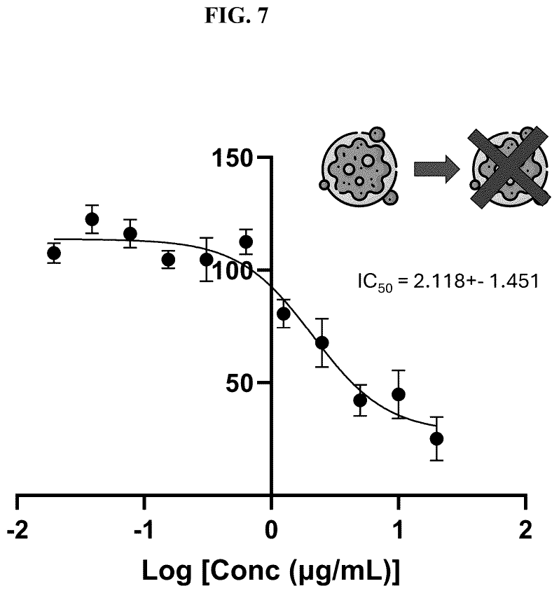

(PDod)3Bi₂I₉ showed excellent activity against pulmonary cancer strains, with an IC₅₀ value of 2.118±1.4 μg/ml, as shown in FIG. 7. This highlights the compound's therapeutic potential and establishes its efficacy in a biological context; the observed cytotoxicity places (PDod)3Bi₂I₉ among a growing class of metal halide-based candidates for targeted cancer treatment. While the material is photoreactive, the cytotoxic mechanism in cells does not depend on photoinduced radical generation. The correlation between dye degradation rates and selective cytotoxicity showed a charge-transfer-driven Type I ROS mechanism rather than singlet oxygen generation characteristic of traditional phototherapeutic systems. While light responsiveness is confirmed electrochemically and through abiotic ROS generation, biological cytotoxicity is predominantly redox-driven under physiological conditions, supporting an intrinsic dark cytotoxicity mechanism (e.g., direct redox cycling, mitochondrial perturbation, or ion imbalance). Comparison screenings under dark conditions against bacterial and fungal strains indicated no activity against non-target species, suggesting selective toxicity toward cancer cells and minimal off-target effects (Table 1).

What is claimed is:

1. A water-stable lead-free bismuth halide semiconductor material comprising: (a) an inorganic bismuth halide framework comprising Bi(III) and a halide X selected from the group consisting of iodide, bromide, chloride, and combinations thereof, the framework comprising Bi₂X₉ dimers that are spatially separated from one another; and (b) an organic phosphonium cationic component that charge-balances the inorganic bismuth halide framework, wherein the material is water-stable and is redox-active to generate reactive oxygen species in an aqueous medium.

2. The material of claim 1, wherein the Bi₂X₉ dimers are face-sharing dimers and the material is zero-dimensional such that the Bi₂X₉ dimers are separated by the organic phosphonium cationic component.

3. The material of claim 1, wherein the organic phosphonium cationic component comprises a trialkylphosphonium cation, a triarylphosphonium cation, an alkylarylphosphonium cation, or a substituted variant thereof.

4. The material of claim 1, wherein the organic phosphonium cationic component comprises at least one C₄-C₂₀ alkyl substituent.

5. The material of claim 1, wherein the material remains crystalline after immersion in liquid water for at least 1 month.

6. The material of claim 1, wherein the material exhibits optical absorption and emission in a visible to near-infrared region.

7. The material of claim 1, wherein the material exhibits an optical absorption edge corresponding to a bandgap in a range from about 1.7 eV to about 3 eV.

8. The material of claim 1, wherein the reactive oxygen species comprises Type I or Type II reactive oxygen species.

9. The material of claim 8, wherein the Type I reactive oxygen species comprises superoxide, hydroxyl radicals, hydroperoxyl radicals, hydrogen peroxide, or a combination thereof, and Type-II reactive oxygen species comprises singlet oxygen species.

10. The material of claim 1, wherein cyclic voltammetry under illumination exhibits increased anodic and cathodic current relative to a dark condition, and wherein electrochemical impedance spectroscopy under illumination exhibits reduced charge-transfer resistance relative to the dark condition.

11. The material of claim 1, wherein Type I reactive oxygen species generation is evidenced by oxidation of TMPD under visible-light exposure with minimal change in an ABDA singlet-oxygen probe under comparable conditions.

12. The material of claim 1, wherein the material comprises (PDod)3Bi₂I₉.

13. A composition comprising the material of claim 1 and water.

14. A method of generating Type I reactive oxygen species in water, comprising contacting the water with the material of claim 1 and illuminating the material with visible light or Visible-NIR light.

15. The method of claim 14, wherein Type I reactive oxygen species generation is evidenced by oxidation of TMPD under visible-light exposure with minimal change in an ABDA singlet-oxygen probe under comparable conditions.

16. The method of claim 14, wherein oxidation of TMPD occurs within about 90 seconds of visible-light exposure as evidenced by emergence of absorbance peaks around 563 nm and 611 nm.

17. The method of claim 14, wherein the method is performed in pure water without addition of an oxidant or a sacrificial electron donor.

18. The method of claim 14, further comprising contacting the water with methylene blue or rhodamine B and monitoring degradation of the methylene blue and/or rhodamine B under illumination.

19. The method of claim 18, wherein the degradation exhibits pseudo-first-order kinetics.

20. The method of claim 14, wherein the visible light or Visible-NIR light has a wavelength from about 450 nm to about 900 nm.
